(19) United States
(12) Patent Application Publication
(10) Pub. No.: US 2018/0008216 A1
(43) Pub. Date: Jan. 11, 2018

(54) X-RAY DETECTOR, AND X-RAY IMAGING APPARATUS HAVING THE SAME

(71) Applicant: Samsung Electronics Co., Ltd., Gyeonggi-do (KR)

(72) Inventors: Dae Soo Kim, Yongin-si (KR); Kyoung Choul Jang, Suwon-si (KR); Jin-Ho Choi, Anyang-si (KR); Seong Hwa Park, Suwon-si (KR)

(21) Appl. No.: 15/644,642

(22) Filed: Jul. 7, 2017

(30) Foreign Application Priority Data
Jul. 7, 2016 (KR) 10-2016-0085944
Jan. 13, 2017 (KR) 10-2017-0006089

(51) Int. Cl. A61B 6/00; A61B 6/10

(52) U.S. Cl. CPC A61B 6/4283; A61B 6/102; A61B 6/4464

(57) ABSTRACT

Disclosed herein are an X-ray detector having impact resistance, and an X-ray imaging apparatus including the same. An X-ray detector for detecting X-rays irradiated from an X-ray source includes a case having at least one opening and a sensing panel configured to convert the X-rays irradiated from the X-ray source into an electrical signal. A frame detachably inserted into the inside of the case through the at least one opening. The frame includes a body on which the sensing panel is disposed and a plurality of legs extending from the edges of the body in a first direction of the X-ray detector. A plurality of buffer members disposed between the plurality of legs and the case and in close contact with the plurality of legs and the case while surrounding a plurality of surfaces of each of the plurality of legs.

### CROSS-REFERENCE TO RELATED APPLICATION(S) AND CLAIM OF PRIORITY

This application claims the benefit of Korean Patent Applications No. 10-2016-0085944, filed on Jul. 7, 2016, and No. 10-2017-0006089, filed on Jan. 13, 2017, in the Korean Intellectual Property Office, the disclosures of which are incorporated herein by reference.

### TECHNICAL FIELD

Embodiments of the present disclosure relate to an X-ray detector and an X-ray imaging apparatus including the same. More particularly, to an X-ray detector having impact resistance and an X-ray imaging apparatus including the same.

### BACKGROUND

An X-ray imaging apparatus is equipment to acquire images of the inside of an object using X-rays. The X-ray imaging apparatus images the inside of an object using a non-invasive method of irradiating X-rays onto the object and detecting X-rays transmitted through the object. Accordingly, a medical X-ray imaging apparatus is used to diagnose an internal injury or a disease of an object that cannot be examined outwardly.

The X-ray imaging apparatus includes an X-ray source to generate X-rays and to irradiate the X-rays onto an object, and an X-ray detector to detect X-rays transmitted through the object. In order to image various parts of an object, the X-ray source is configured to be movable. More specifically, the X-ray detector is used in a table mode when it is installed in a radiography table, in a stand mode when it is installed in a radiography stand, and in a portable mode when it is not fixed at a specific location.

However, when an external impact is applied to the X-ray detector, the performance of the X-ray detector may deteriorate. More specifically, when an external impact is directly transferred to a vulnerable portion, such as a sensing panel or a circuit board, the vulnerable portion may be broken or malfunction, resulting in deterioration in performance of the X-ray detector.

### SUMMARY

To address the above-discussed deficiencies, it is a primary object to provide an X-ray detector having an improved structure capable of preventing a sensing panel from being broken by an external impact, and an X-ray imaging apparatus including the X-ray detector.

Additional aspects of the disclosure will be set forth in part in the description which follows and, in part, will be obvious from the description, or may be learned by practice of the disclosure.

In accordance with one aspect of the present disclosure, an X-ray detector of detecting X-rays irradiated from an X-ray source includes a case having at least one opening, a sensing panel configured to convert the X-rays irradiated from the X-ray source into an electrical signal, a frame detachably inserted into the inside of the case through the at least one opening, the frame including a body on which the sensing panel is disposed, and a plurality of legs extending from the edges of the body in thickness directions of the X-ray detector, and a plurality of buffer members disposed between the plurality of legs and the case in such a way to be in close contact with the plurality of legs and the case, while surrounding a plurality of surfaces of each of the plurality of legs.

The plurality of buffer members surround a plurality of surfaces of the plurality of legs, the surfaces facing the case.

Each of the plurality of legs includes a first surface facing one direction of the thickness directions of the X-ray detector, a second surface facing the other direction of the thickness directions of the X-ray detector, and a third surface connecting the first surface to the second surface, and facing the case. The plurality of buffer members are disposed to surround the first surface, the second surface, and the third surface.

The sensing panel is disposed on the body in such a way to be spaced apart from the frame.

The body includes a sensing panel corresponding surface facing the sensing panel. In accordance with one aspect of the present disclosure, the X-ray detector further includes at least one support member disposed between the sensing panel and the sensing panel corresponding surface, and having an elastic material.

In accordance with one aspect of the present disclosure, the X-ray detector further includes a plurality of cushion members disposed between the plurality of legs and the sensing panel, and having an elastic material.

In accordance with one aspect of the present disclosure, the X-ray detector further includes at least one cap detachably coupled with the at least one opening to open or close the at least one opening of the case.

The case has a plurality of openings that are opposite to each other in a direction in which the frame is inserted. In accordance with one aspect of the present disclosure, the X-ray detector further includes a plurality of caps configured to open or close the plurality of openings of the case, and one cap of the plurality of caps is integrated into the frame.

In accordance with one aspect of the present disclosure, an X-ray detector of detecting X-rays includes a case having at least one opening, a frame detachably inserted into the inside of the case through the at least one opening, a sensing panel configured to convert the X-rays into an electrical signal, and disposed on the frame in such a way to be spaced apart from the case and the frame, and a buffer member configured to prevent the frame from directly contacting the case, and disposed between the case and the frame in such a way to be in close contact with the case and the frame, while surrounding a plurality of surfaces of the frame.

The frame includes a body on which the sensing panel is disposed, and a plurality of legs extending from the edges of the body in thickness directions of the X-ray detector. The buffer member is disposed between the case and the plurality of legs in such a way to be in close contact with the case and the plurality of legs, while surrounding a plurality of surfaces of each of the plurality of legs.

The case includes a first frame forming an incident surface onto which the X-rays are incident, a second frame facing the first frame and a plurality of third frames forming an outer appearance of the case, together with the first frame and the second frame. The buffer member surrounds a plurality of surfaces of the frame in such a way to be in close contact with the first frame, the second frame, and the plurality of third frames.

In accordance with one aspect of the present disclosure, the X-ray detector further includes a support member having at least one part disposed between the sensing panel and the body, and having an elastic material.

An incision is formed in the body, and a part of the support member is inserted into the incision.

In accordance with one aspect of the present disclosure, the X-ray detector further includes a cushion member disposed between the plurality of legs and the sensing panel along the edges of the sensing panel, and having an elastic material.

In accordance with one aspect of the present disclosure, the X-ray detector further includes at least one cap configured to open or close the at least one opening of the case.

In accordance with one aspect of the present disclosure, an X-ray detector of detecting X-rays includes a case, a frame disposed in the inside of the case in such a way to be spaced apart from the case, a sensing panel configured to convert the X-rays into an electrical signal, and disposed on the frame in such a way to be spaced apart from the case and the frame, and an impact absorbing member disposed in the inside of the case, and configured to absorb an external impact. The impact absorbing member includes a first impact absorbing member disposed between the frame and the case, and a second impact absorbing member disposed between the frame and the sensing panel to be positioned in the inside of the first impact absorbing member in a width direction of the X-ray detector.

The first impact absorbing member is disposed between the case and the frame in such a way to be in close contact with the case and the frame, while surrounding a plurality of surfaces of the frame.

The case includes a first frame forming an incident surface onto which the X-rays are incident, a second frame facing the first frame, and a plurality of third frames forming an outer appearance of the case, together with the first frame and the second frame. The first impact absorbing member surrounds the plurality of surfaces of the frame in such a way to be in close contact with the first frame, the second frame, and the plurality of third frames.

The impact absorbing member further includes a third impact absorbing member having at least one part disposed between the frame and the sensing panel in thickness directions of the X-ray detector.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a perspective view of an X-ray imaging apparatus according to an embodiment of the present disclosure;

FIG. 2 illustrates a view for describing an operation principle of a sensing panel of the X-ray imaging apparatus according to the embodiment of the present disclosure;

FIG. 3 illustrates a perspective view of an X-ray detector according to a first embodiment of the present disclosure;

FIG. 4 illustrates an exploded perspective view showing some components of the X-ray detector according to the first embodiment of the present disclosure;

FIG. 5 illustrates an exploded perspective view of a portion “A” of FIG. 4;

FIG. 6 illustrates a cross-sectional view of the X-ray detector of FIG. 3, taken along line C-C of FIG. 3;

FIG. 7 illustrates an exploded perspective view showing some components of an X-ray detector according to a second embodiment of the present disclosure;

FIG. 8 illustrates a perspective view of an X-ray detector according to a third embodiment of the present disclosure;

FIG. 9 illustrates an exploded perspective view of the X-ray detector according to the third embodiment of the present disclosure;

FIG. 10 illustrates a cross-sectional view of the X-ray detector of FIG. 8, taken along line I-I′;

FIG. 11A illustrates an enlarged cross-sectional view of a portion surrounded by a right circle in the X-ray detector of FIG. 10;

FIG. 11B illustrates an enlarged cross-sectional view of a portion surrounded by a left circle in the X-ray detector of FIG. 10;

FIG. 12 illustrates an exploded perspective view showing some components of an X-ray detector according to a fourth embodiment of the present disclosure;

FIG. 13 illustrates an exploded perspective view of a portion P of FIG. 12;

FIG. 14 illustrates a cross-sectional view of the X-ray detector of FIG. 12;

FIG. 15 illustrates an exploded perspective view showing some components of an X-ray detector according to a fifth embodiment of the present disclosure.

### DETAILED DESCRIPTION

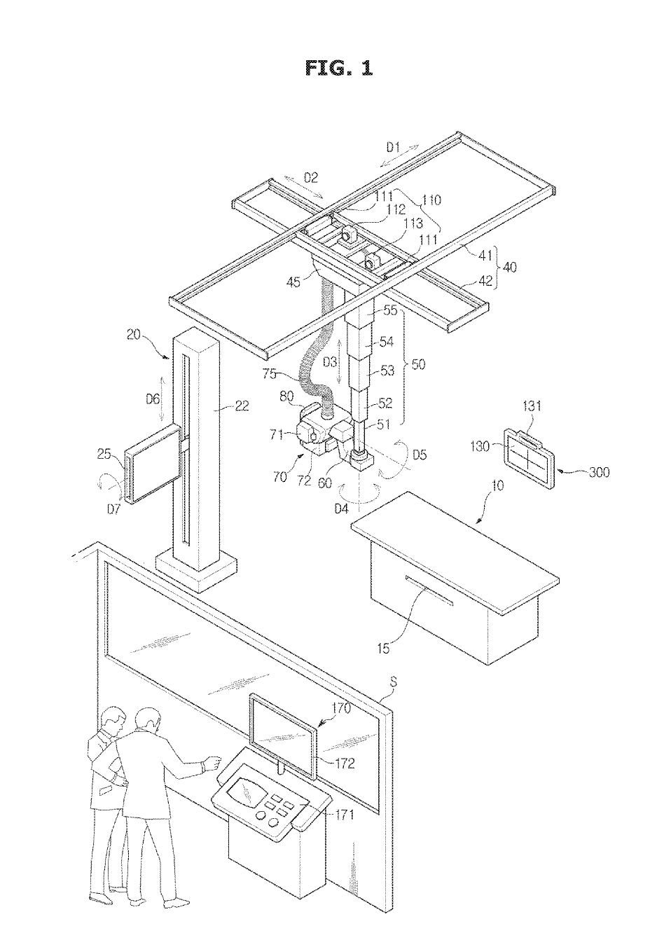

FIGS. 1 through 15, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

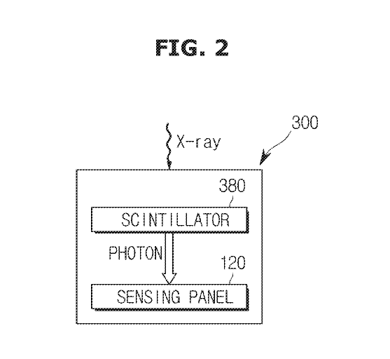

In FIGS. 1 and 2, a case to which an X-ray detector according to a first embodiment of the present disclosure is applied will be described as an example.

FIG. 1 illustrates a perspective view of an X-ray imaging apparatus according to an embodiment of the present disclosure, and FIG. 2 is a view for describing an operation principle of a sensing panel of the X-ray imaging apparatus according to the embodiment of the present disclosure. FIG. 2 shows the X-ray detector to which an indirect conversion mode is applied.

As shown in FIG. 1, an X-ray imaging apparatus 1 may include one or more guide rails 40, a carriage 45, a post frame 50, a motor 110, an X-ray source 70, an X-ray detector 300, a manipulator 80, and a workstation 170. The X-ray imaging apparatus 1 may further include a radiography table 10 and a radiography stand 20 in which the X-ray detector 300 can be installed.

The guide rails 40, the carriage 45, and the post frame 50 may be used to move the X-ray source 70 toward an object.

The guide rails 40 may include a first guide rail 41 and a second guide rail 42 arranged to form a predetermined angle with respect to each other. The first guide rail 41 may be orthogonal to the second guide rail 42.

The first guide rail 41 may be installed on a ceiling of an examination room where the X-ray imaging apparatus 1 is placed.

The second guide rail 42 may be disposed beneath the first guide rail 41, and slide with respect to the first guide rail 41. The first guide rail 41 may include a plurality of rollers (not shown) that are movable along the first guide rail 41. The second guide rail 42 may connect to the rollers and move along the first guide rail 41.

A direction in which the first guide rail 41 extends is defined as a first direction D1, and a direction in which the second guide rail 42 extends is defined as a second direction D2. Accordingly, the first direction D1 may be orthogonal to the second direction D2, and the first and second directions D1 and D2 may be parallel to the ceiling of the examination room.

The carriage 45 may be disposed beneath the second guide rail 42, and move along the second guide rail 42. The carriage 45 may include a plurality of rollers (not shown) to move along the second guide rail 42. Accordingly, the carriage 45 may be movable in the first direction D1 together with the second guide rail 42, and movable in the second direction D2 along the second guide rail 42. The post frame 50 may be fixed on the carriage 45 and disposed below the carriage 45. The post frame 50 may include a plurality of posts 51, 52, 53, 54, and 55.

The posts 51, 52, 53, 54, and 55 may connect to each other such that they can be folded with each other. The length of the post frame 50 fixed on the carriage 45 may increase or decrease in the elevation direction of the examination room.

A direction in which the length of the post frame 50 increases or decreases is defined as a third direction D3. Accordingly, the third direction D3 may be orthogonal to the first direction D1 and the second direction D2.

The X-ray source 70 may irradiate X-rays to an object. Herein, the object may be a human's or animal's living body, however, the object is not limited to these. That is, the object may be anything whose inside structure can be imaged by the X-ray imaging apparatus 1.

The X-ray source 70 may include an X-ray tube 71 to generate X-rays, and a collimator 72 to guide the generated X-rays to be headed toward an object.

A revolute joint 60 may be disposed between the X-ray source 70 and the post frame 50.

The revolute joint 60 may couple the X-ray source 70 with the post frame 50, and support a load applied to the X-ray source 70.

The revolute joint 60 may include a first revolute joint (not shown) connected to the lower post 51 of the post frame 50, and a second revolute joint (not shown) connected to the X-ray source 70.

The first revolute joint may be rotatable with respect to the central axis of the post frame 50 extending in the elevation direction of the examination room. Accordingly, the first revolute joint may rotate on a plane that is perpendicular to the third direction D3. The rotation direction of the first revolute joint is defined as a fourth direction D4, and the fourth direction D4 is a rotation direction of an axis parallel to the third direction D3.

The second revolute joint may be rotatable on a plane that is perpendicular to the ceiling of the examination room. Accordingly, the second revolute joint may rotate in a rotation direction of an axis parallel to the first direction D1 and the second direction D2. The rotation direction of the second rotation joint 62 is defined as a fifth direction D5, and the fifth direction D5 is a rotation direction of an axis extending in the first direction D1 or the second direction D2. The X-ray source 70 may connect to the revolute joint 60 and rotate in the fourth direction D4 and the third direction D5. Also, the X-ray source 70 may connect to the post frame 50 through the revolute joint 60, and linearly move in the first direction D1, in the second direction D2, or in the third direction D3.

In order to move the X-ray source 70 in the first direction D1 through the fifth direction D5, the motor 110 may be used. The motor 110 may be electrically driven, and may include an encoder.

The motor 110 may include a first motor 111, a second motor 112, a third motor 113, a fourth motor (not shown), and a fifth motor (not shown) to correspond to the respective directions.

The first to fifth motors 111 to 115 may be arranged at appropriate locations in consideration of convenience of design. For example, the first motor 111 that is used to move the second guide rail 42 in the first direction D1 may be disposed around the first guide rail 41, the second motor 112 that is used to move the carriage 45 in the second direction D2 may be disposed around the second guide rail 42, and the third motor 113 that is used to increases or decreases the length of the post frame 50 in the third direction D3 may be disposed in the carriage 45. Also, the fourth motor 114 that is used to rotate the X-ray source 70 in the fourth direction D4 may be disposed around the first revolute joint 61, and the fifth motor 115 that is used to rotate the X-ray source 70 in the fifth direction D5 may be disposed around the second revolute joint.

The motor 110 may connect to power transfer means (not shown) in order to linearly move or rotate the X-ray source 70 in the first to fifth directions D1 to D5. The power transfer means may be a belt and a pulley, a chain and a sprocket, or a shaft.

In one side of the X-ray source 70, the manipulator 80 may be disposed to provide a user interface. The user is a person who diagnoses an object using the X-ray imaging apparatus 1, and may be a medical staff including a doctor, a radiological technologist, and a nurse. However, the user is not limited to the above-mentioned persons, and may be anyone using the X-ray imaging apparatus 1.

The X-ray detector 300 may detect X-rays transmitted through an object. In the front side of the X-ray detector 300, an incident surface 130 onto which X-rays are incident may be provided, and a sensing panel 120 (see FIG. 2) may be installed in the X-ray detector 100. In the sensing panel 120, a plurality of pixels 150 (see FIG. 2) that respond to X-rays may be arranged in a matrix form. In the upper center portion of the X-ray detector 300, a handle 131 may be provided so that the user can move or carry the X-ray detector 300.

The X-ray detector 300 may operate in various radiography modes according to a position of the X-ray detector 300 upon radiography. More specifically, the X-ray detector 300 may operate in a table mode when it is installed in the radiography table 10, in a stand mode when it is installed in the radiography stand 20, or in a portable mode when it is placed at an arbitrary location according to a position of an object or a part of an object to be scanned, instead of the radiography table 10 or the radiography stand 20. Particularly, accommodating slots 15 and 25 into which the X-ray detector 100 can be inserted may be formed in the radiography table 10 and the radiography stand 20. The accommodating slot 15 formed in the radiography table 10 is defined as a first accommodating slot 15, and the accommodating slot 25 formed in the radiography stand 20 is defined as a second accommodating slot 25. The second accommodating slot 25 may be movable in the length direction of a support bar 22, and rotatable in the rotation direction of an axis perpendicular to the length direction of the support bar 22, as illustrated in FIG. 1. The length direction of the support bar 22 is defined as a sixth direction D6, and the rotation direction of the axis perpendicular to the sixth direction D6 is defined as a seventh direction D7.

The workstation 170 may include an input device 171 and a second display 172 to provide a user interface, like the manipulator 80. Accordingly, the user can input various kinds of information for radiography or manipulate individual devices through the workstation 170. Also, the user may input various commands (e.g., a command for selecting a radiography location, a start command for radiography, etc.) related to operations of the X-ray imaging apparatus 1 through the workstation 170. In addition, the user may examine images acquired during radiography through the workstation 170.

The input device 171 may include at least one of a switch, a keyboard, a trackball, a mouse, and a touch screen, for example. If the input device 171 is implemented as a Graphic User Interface (GUI) such as a touch screen, in other words, if the input device 171 is implemented in software, the input device 171 may be displayed through the second display 172. The second display 172 may be, like the first display (not shown), implemented as a CRT, a LCD, or a LED display, although not limited to these.

The workstation 170 may include various processors, such as a Central Processing Unit (CPU) or a Graphic Processing Unit (GPU), and a Printed Circuit Board (PCB), and may further include various kinds of storage devices as necessary. Accordingly, the workstation 170 may include main components (e.g., a controller (not shown)) of the X-ray imaging apparatus 1 to make determinations for operations of the X-ray imaging apparatus 1 or to generate various control signals.

The workstation 170 may be placed in independent space S where X-rays can be blocked, and may be connected to the X-ray source 70 and the X-ray detector 300 through wired/wireless communication.

The X-ray source 70 may be used to generate X-rays and to irradiate the X-rays on an object, and may include an X-ray tube 71 to generate X-rays.

The X-ray detector 300 may be used to detect X-rays irradiated from the X-ray source 70 and then transmitted through an object. The X-rays may be detected by the sensing panel 120 installed in the X-ray detector 300. The sensing panel 120 may convert the detected X-rays into electrical signals, and acquire an image about the inside of the object.

The sensing panel 120 can be classified according to its material configuration, a method of converting detected X-rays into electrical signals, and a method of acquiring image signals.

The sensing panel 120 is classified into a mono type device or a hybrid type device according to its material configuration.

If the sensing panel 120 is a mono type device, a portion of detecting X-rays and generating electrical signals, and a portion of reading and processing the electrical signals may be semiconductors made of the same material, or may be manufactured by one process. In this case, the sensing panel 120 may be a Charge Coupled Device (CCD) or a Complementary Metal Oxide Semiconductor (CMOS) which is a light receiving device.

If the sensing panel 120 is a hybrid type device, a portion of detecting X-rays and generating electrical signals, and a portion of reading and processing the electrical signals may be made of different materials, or may be manufactured by different processes. For example, there are cases of detecting X-rays using a light receiving device, such as a photodiode, a CCD, or CdZnTe, and reading and processing electrical signals using a CMOS Read Out Integrated Circuit (CMOS ROIC), of detecting X-rays using a strip detector, and reading and processing electrical signals using a CMOS ROIC, and of using an a-Si or a-Se flat panel system.

The sensing panel 120 may use a direct conversion mode and an indirect conversion mode according to a method of converting X-rays into electrical signals.

In the direct conversion mode, if X-rays are irradiated, electron-hole pairs are temporarily generated in a light receiving device, electrons move to an anode, and holes move to a cathode by an electric field applied to both terminals of the light receiving device. The sensing panel 120 converts the movements of the electrons and holes into electrical signals. The light receiving device may be made of, for example, a-Se, CdZnTe, HgI2, or PbI2.

As shown in FIG. 2, in the indirect conversion mode, if X-rays irradiated from the X-ray source 70 (see FIG. 1) react with a scintillator 380 to emit photons having a wavelength of a visible light region, the light receiving device detects the photons, and converts the photons into electrical signals. The light receiving device may be made of a-Si, and the scintillator 380 may be a GADOX scintillator of a thin film type, or a CSI (TI) of a micro pillar type or a needle type. In FIG. 2, the sensing panel 120 is used as a light receiving device.

The sensing panel 120 may use a Charge Integration Mode (CIM) of storing charges for a predetermined time period and then acquiring a signal from the stored charges, or a Photon Counting Mode (PCM) of counting the number of photons whenever a signal is generated by a single X-ray photon, according to a method of acquiring electrical signals.

The sensing panel 120 may use any mode among the above-described modes.

The X-ray detector 300 described above may operate in the table mode, in the stand mode, or in the portable mode in order to detect X-rays.

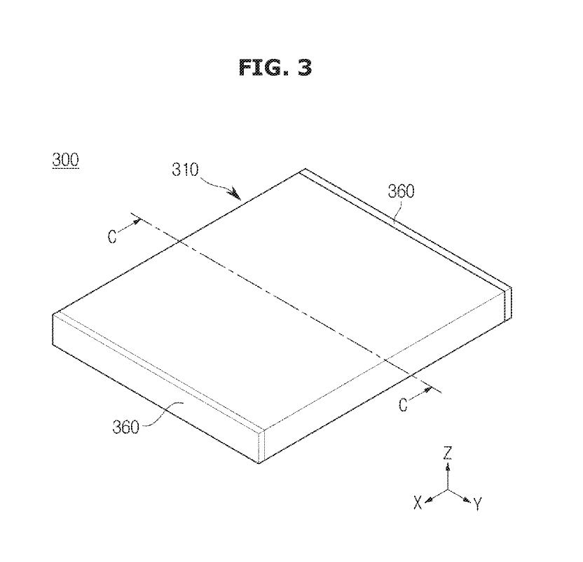

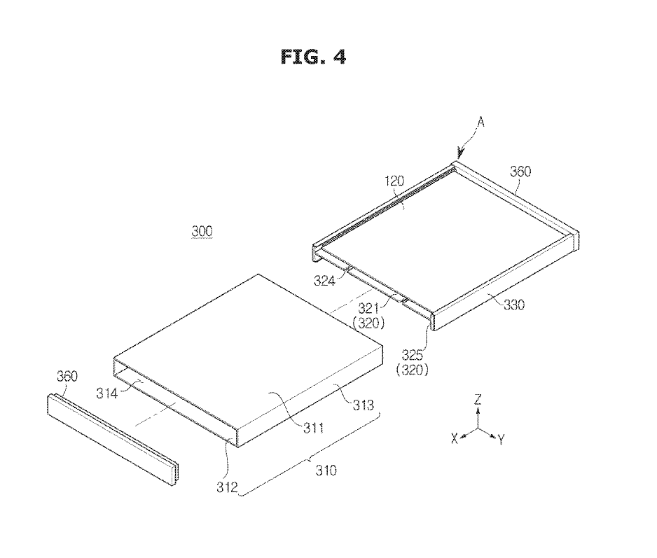

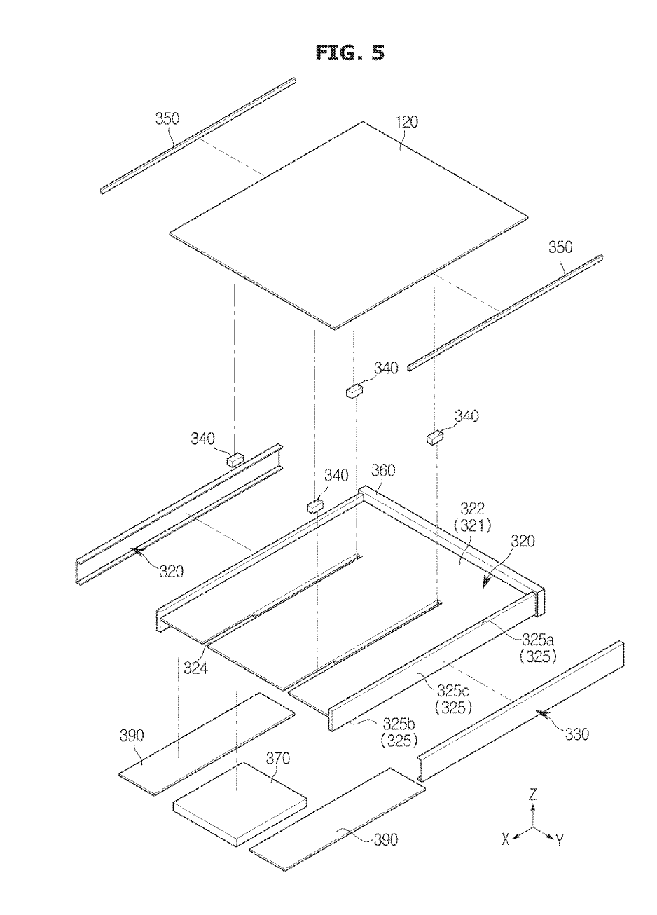

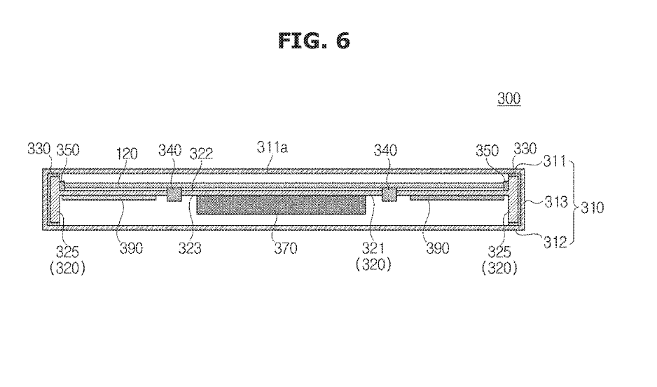

FIG. 3 illustrates a perspective view of an X-ray detector according to a first embodiment of the present disclosure, and FIG. 4 illustrates an exploded perspective view showing some components of the X-ray detector according to the first embodiment of the present disclosure. FIG. 5 illustrates an exploded perspective view of a portion “A” of FIG. 4, and FIG. 6 illustrates a cross-sectional view of the X-ray detector of FIG. 3, taken along line C-C of FIG. 3.

As shown in FIGS. 3 to 6, an X-ray detector 300 may detect X-rays irradiated from the X-ray source 70.

The X-ray detector 300 may include a case 310. The case 310 may form an outer appearance of the X-ray detector 300.

The case 310 may include a first frame 311. On the first frame 311, an incident surface 311a onto which X-rays are incident may be formed.

The case 310 may further include a second frame 312. The second frame 312 may be opposite to the first frame 311.

The case 310 may further include a plurality of third frames 313. The third frames 313 may form the outer appearance of the case 310, together with the first frame 311 and the second frame 312. The plurality of third frames 313 may connect the first frame 311 to the second frame 312. More specifically, the plurality of third frames 313 may connect the first frame 311 to the second frame 312 in thickness directions Z of the X-ray detector 300.

The case 310 may have at least one opening 314. For example, the case 310 of the X-ray detector 300 according to the first embodiment of the present disclosure may have a plurality of openings 314 that are opposite to each other in a direction in which a frame 320 is inserted. The case 310 may be in the shape of a box, wherein a plurality of sides facing each other open.

The case 310 may be formed of Carbon Fiber Reinforced Plastic (CFRP), although not limited to this.

The X-ray detector 300 may further include the frame 320.

The frame 320 may be detachably inserted into the inside of the case 310 through the at least one opening 314.

The frame 320 may be disposed in the inside of the case 310 in such a way to be spaced apart from the case 310. In other words, the frame 320 may be disposed in the inside of the case 310 in such a way not to contact the case 310. As such, since the frame 320 does not directly contact the case 310, an impact or an external force applied to the case 310 may be prevented from being transferred to the frame 320 and the sensing panel 120 disposed on the frame 320.

The frame 320 may include a body 321. The sensing panel 120 may be disposed on the body 321.

The body 321 may include a sensing panel corresponding surface 322 facing the sensing panel 120. The sensing panel corresponding surface 322 of the body 321 may face the first frame 311 of the case 310. The sensing panel 120 may be disposed on the sensing panel corresponding surface 322.

The body 321 may further include a circuit board corresponding surface 323 facing a circuit board 390. The circuit board corresponding surface 323 of the body 321 may face the second frame 312 of the case 310. The circuit board 390 may be disposed on the circuit board corresponding surface 323 of the body 321. Also, a battery 370 may be disposed on the circuit board corresponding surface 323 of the body 321. However, the circuit board 390 and the battery 370 may be disposed at any other locations, instead of the circuit board corresponding surface 323.

The body 321 may include at least one incision 324. The at least one incision 324 may be in the shape of a slit. However, the at least one incision 324 may be formed in any other shape, instead of a slit.

The frame 320 may further include a plurality of legs 325. The plurality of legs 325 may extend from the edges of the body 321 in the thickness directions Z of the X-ray detector 300.

Each of the plurality of legs 325 may include a first surface 325a. The first surface 325a may face one direction of the thickness directions Z of the X-ray detector 300. More specifically, the first surface 325a may face the first frame 311 of the case 310.

Also, each of the plurality of legs 325 may further include a second surface 325b. The second surface 325b may face the other direction of the thickness directions Z of the X-ray detector 300. More specifically, the second surface 325b may face the second frame 312 of the case 310.

Each of the plurality of legs 325 may further include a third surface 325c. The third surface 325c may connect the first surface 325a to the second surface 325b, and face the case 310. More specifically, the third surface 325c may connect the first surface 325a to the second surface 325b, and face one of the plurality of third frames 313.

The frame 320 may have a cross section in the shape of “H” by coupling the body 321 with the plurality of legs 325.

The X-ray detector 300 may further include a buffer member 330. Preferably, the X-ray detector 300 may further include a plurality of buffer members 330. Since the plurality of buffer members 330 have the same structure, one of the buffer members 330 will be described below.

The buffer member 330 may be disposed between the case 310 and the frame 320 in order to prevent an external impact applied to the case 310 from being directly transferred to the frame 320. In other words, the buffer member 330 may be disposed between the case 310 and the frame 320 to first absorb an external impact applied to the case 310. That is, the buffer member 330 may prevent the case 310 from directly contacting the frame 320.

The buffer member 330 may be in close contact with at least one of the case 310 and the frame 320. More specifically, the buffer member 330 may be in close contact with at least one of the opposite inner walls of the case 310 and the frame 320. Preferably, the buffer member 330 may be disposed between the case 310 and the frame 320 in such a way to be in close contact with the case 310 and the frame 320.

The buffer member 330 may surround the plurality of surfaces of the frame 320. More specifically, the buffer member 330 may be disposed between the case 310 and the frame 320 in such a way to be in close contact with the case 310 and the frame 320, while surrounding the plurality of surfaces of the plurality of legs 325.

In other words, the buffer member 330 may surround the plurality of surfaces of the frame 320 to be in close contact with the first frame 311, the second frame 312, and the plurality of third frames 313. More specifically, the buffer member 330 may surround the plurality of surfaces of the plurality of legs 325 in such a way to be in close contact with the first frame 311, the second frame 312, and the plurality of frames 313.

The buffer member 330 may surround the plurality of surfaces of the plurality of legs 325 facing the case 310. More specifically, the buffer member 330 may surround the first surfaces 325a, the second surfaces 325b, and the third surfaces 325c of the plurality of legs 325. In this case, the buffer member 330 may have a cross section in the shape of a rectangle whose one side opens.

In other words, the buffer member 330 may surround the plurality of surfaces of the plurality of legs 325 that may contact the case 310. Generally, the edges of the sensing panel 120 may be vulnerable to impacts. If the buffer member 330 is disposed in this way, it is possible to effectively prevent an external impact applied to the edges of the X-ray detector 300 from being transferred to the sensing panel 120, thereby reducing a degree of breakage or damage of the sensing panel 120 due to the external impact.

The X-ray detector 300 may further include the sensing panel 120. The sensing panel 120 may be used to convert X-rays irradiated by the X-ray source 70 into electrical signals.

The sensing panel 120 may include a plurality of pixels (not shown), and each pixel may include a thin-film transistor and a photoelectric conversion device such as a photodiode. The sensing panel 120 may read out the intensity of light passed through the scintillator 380 (see FIG. 2) for each pixel. The sensing panel 120 may include an electrical circuit to transfer outputs from the photoelectric conversion device to the outside.

The sensing panel 120 may be spaced apart from at least one of the frame 320 and the case 310. Preferably, the sensing panel 120 may be disposed on the frame 320 in such a way to be spaced apart from the frame 320 and the case 310.

The sensing panel 120 may be disposed on the body 321 in such a way to be spaced apart from the frame 320. In other words, the sensing panel 120 may be disposed on the body 321 in such a way not to directly contact the frame 320. More specifically, the sensing panel 120 may be disposed on the sensing panel corresponding surface 322 in such a way to be spaced apart from the sensing panel corresponding surface 322.

The X-ray detector 300 may further include the scintillator 380 (see FIG. 2). The scintillator 380 may include a phosphor. The scintillator 380 may convert incident X-rays into visible light. On one surface of the scintillator 380, a cover (not shown) may be disposed to protect the scintillator 380. The scintillator 380 may be formed of a metal material such as aluminum.

The X-ray detector 300 may further include the circuit board 390.

The circuit board 390 may perform operation for acquiring an image of an object using data acquired based on signals read out by the sensing panel 120. The circuit board 390 may be accommodated in the inside of the X-ray detector 300 in order to control driving of the X-ray detector 300. The circuit board 390 may include memory and an operator. The memory may store shadow information of an object according to an incidence angle of X-rays, and the operator may calculate an incidence angle of X-rays based on a shadow shape of an object formed on the sensing panel 120 and shadow information stored in the memory. The memory and the operator may be disposed outside the X-ray detector 300.

The sensing panel 120 may be electrically connected to the circuit board 390.

The X-ray detector 300 may further include the at least one support member 340.

The at least one support member 340 may be disposed between the sensing panel 120 and the body 321. In other words, the at least one support member 340 may be disposed between the sensing panel 120 and the sensing panel corresponding surface 322.

At least one of the at least one support member 340 may be disposed between the sensing panel 120 and the body 321. For example, a part of the support member 340 may be inserted into the incision 324 such that the part of the support member 340 is located between the sensing panel 120 and the body 321.

If the X-ray detector 300 includes a plurality of support members 340, a part of the plurality of support members 340 may be coupled with at least one of the sensing panel 120 and the body 321 to be located between the sensing panel 120 and the body 321, and the other part of the plurality of support members 340 may be inserted into the incision 324 to be located between the sensing panel 120 and the body 321.

The at least one support member 340 may be formed of an elastic material. The elastic material may be rubber, silicon, or the like, although not limited to these.

The at least one support member 340 may prevent the sensing panel 120 from directly contacting the frame 320 in the thickness directions Z of the X-ray detector 300, thereby preventing an external impact from being transferred to the sensing panel 120 through the frame 320. That is, the at least one support member 340 may absorb an external impact transferred to the frame 320 to prevent breakage or damage of the sensing panel 120 due to the external impact.

The X-ray detector 300 may further include a cushion member 350.

The cushion member 350 may be disposed between the plurality of legs 325 and the sensing panel 120. More specifically, the cushion member 350 may be disposed between the plurality of legs 325 and the sensing panel 120 along the edges of the sensing panel 120.

The cushion member 350 may be formed of an elastic material. The elastic material may be rubber, silicon, or the like, although not limited to these.

The cushion member 350 may prevent the sensing panel 120 from directly contacting the frame 320, specifically, the plurality of legs 325 in the width direction Y of the X-ray detector 300, thereby preventing an external impact from being transferred to the sensing panel 120 through the plurality of legs 325. That is, the cushion member 350 may absorb an external impact transferred to the frame 320, specifically, to the plurality of legs 325, thereby preventing breakage or damage of the sensing panel 120 due to the external impact.

The X-ray detector 300 may further include the at least one cap 360.

The at least one cap 360 may open or close the at least one opening 314 of the case 310.

Also, the at least one cap 360 may be detachably coupled with the at least one opening 314.

The X-ray detector 300 according to the first embodiment of the present disclosure may include the plurality of caps 360 configured to open or close the plurality of openings 314 of the case 310. The plurality of openings 314 may be formed to be opposite to each other in the direction X in which the frame 320 is inserted. Any one of the plurality of caps 360 may be integrated into the frame 320. Or, any one of the plurality of caps 360 may be coupled with the frame 320. More specifically, any one of the plurality of caps 360 may be integrated into the frame 320 to connect one ends of the plurality of legs 325 in the width direction Y of the X-ray detector 300. Also, any one of the plurality of caps 360 may be coupled with the frame 320 to connect one ends of the plurality of legs 325 in the width direction Y of the X-ray detector 300.

If the X-ray detector 300 includes a single cap 360, the case 310 may include a single opening 314. In this case, the case 310 may be in the shape of a box whose one side opens.

The X-ray detector 300 may further include the battery 370.

The battery 370 may be installed in the inside of the case 310.

The battery 370 may face the circuit board corresponding surface 323 of the body 321. Alternatively, the battery 370 may be disposed on the circuit board corresponding surface 323 of the body 321. However, the position of the battery 370 is not limited to this, and the battery 370 may be positioned at any other location.

The X-ray detector 300 may further include the terminal (not shown) with which the coupling module (not shown) is coupled. The terminal may be disposed in the X-ray detector 300 such that the coupling module can be coupled with the terminal. In other words, the terminal may be disposed in the X-ray detector 300 such that the coupling module electrically connected to the circuit board 390 can be coupled with the terminal. That is, the coupling module may be coupled with the terminal to be electrically connected to the circuit board 390. The terminal may be disposed at one side of the case 310, although not limited to this. That is, the terminal may be disposed at any other location.

Now, the configuration of the X-ray detector 300 will be described in another aspect based on an impact absorbing member. Hereinafter, the same description as that described above will be omitted.

The X-ray detector 300 may include the case 310. The case 310 may form the outer appearance of the X-ray detector 300. The case 310 may include the first frame 311 forming the incident surface 311a onto which X-rays are incident. Also, the case 310 may further include the second frame 312 facing the first frame 311. Also, the case 310 may further include the plurality of third frames 313 forming the outer appearance of the case 310, together with the first frame 311 and the second frame 312. The plurality of third frames 313 may connect the first frame 311 to the second frame 312 in the thickness directions Z of the X-ray detector 300.

The X-ray detector 300 may further include the frame 320. The frame 320 may be installed in the case 310 in such a way to be spaced apart from the case 310.

The X-ray detector 300 may further include the sensing panel 120. The sensing panel 120 may convert X-rays into electrical signals. The sensing panel 120 may be installed in the inside of the case 310. Also, the sensing panel 120 may be disposed on the frame 320 in such a way to be spaced apart from the case 310 and the frame 320.

The X-ray detector 300 may further include an impact absorbing member. The impact absorbing member may be installed in the inside of the case 310 to absorb an external impact.

The impact absorbing member may be formed of an elastic material. The elastic material may be rubber, silicon, or the like, although not limited to these.

The impact absorbing member may include a first impact absorbing member 330. The first impact absorbing member 330 may be disposed between the frame 320 and the case 310.

The impact absorbing member may further include a second impact absorbing member 350. The second impact absorbing member 350 may be disposed between the frame 320 and the sensing panel 120. More specifically, the second impact absorbing member 350 may be disposed between the frame 320 and the sensing panel 120 to be positioned in the inside of the first impact absorbing member 330 in the width direction Y of the X-ray detector 300. In other words, the impact absorbing member may be disposed in a dual structure in the width direction Y of the X-ray detector 300 in the inside of the X-ray detector 300 in order to prevent an external impact applied to the X-ray detector 300 from being transferred to the sensing panel 120. Generally, the edges of the X-ray detector 300 may be vulnerable to external impacts. The first impact absorbing member 330 located at the outer position in the width direction Y of the X-ray detector 300 may mainly block an external impact, and the second impact absorbing member 350 located in the inside of the first impact absorbing member 330 in the width direction Y of the X-ray detector 300 may secondarily block the external impact. Through the structure, components of the X-ray detector 300, such as the sensing panel 120, can be prevented from being damaged or broken by an external impact transferred in the width direction Y of the X-ray detector 300.

The first impact absorbing member 330 may be disposed between the case 310 and the frame 320 in such a way to be in close contact with the case 310 and the frame 320, while surrounding the plurality of surfaces of the frame 320. In other words, the first impact absorbing member 330 may surround the plurality of surfaces of the frame 320 to be in close contact with the first frame 311, the second frame 312, and the plurality of third frames 313.

The impact absorbing member may further include the third impact absorbing member 340. At least one part of the third impact absorbing member 340 may be disposed between the frame 320 and the sensing panel 120 in the thickness directions Z of the X-ray detector 300. The third impact absorbing member 340 may supplement the first impact absorbing member 330 and the second impact absorbing member 350 to prevent components (for example, the sensing panel 120) of the X-ray detector 300 from being damaged or broken by an external impact transferred in the thickness directions Z of the X-ray detector 300, as well as an external impact transferred in the width direction Y of the X-ray detector 300. Through the structure, it is possible to effectively block external impacts, resulting in an improvement of the impact resistance of the X-ray detector 300.

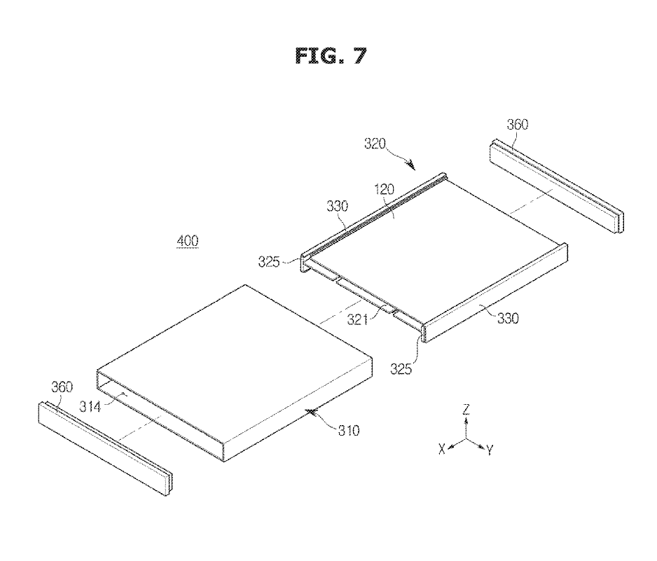

FIG. 7 illustrates an exploded perspective view showing some components of an X-ray detector according to a second embodiment of the present disclosure. Hereinafter, the same description as that described above with reference to FIGS. 3 to 6 will be omitted.

As shown in FIG. 7, an X-ray detector 400 may include the plurality of caps 360.

The plurality of caps 360 may open or close the plurality of openings 314 formed to be opposite to each other in the direction X in which the frame 320 is inserted.

The plurality of caps 360 may be detachably coupled with the plurality of openings 314. That is, any one of the plurality of caps 360 may be detachably coupled with any one of the plurality of openings 314 to open or close the one of the plurality of openings 314, and the other one of the plurality of caps 360 may be detachably coupled with the other one of the plurality of openings 314 to open or close the other one of the plurality of openings 314.

The plurality of caps 360 may be separated from the frame 320.

If the X-ray detector 400 includes the plurality of caps 360, the plurality of openings 314 may be formed in the case 310. In this case, the case 310 may be in the shape of a box, wherein a plurality of sides open.

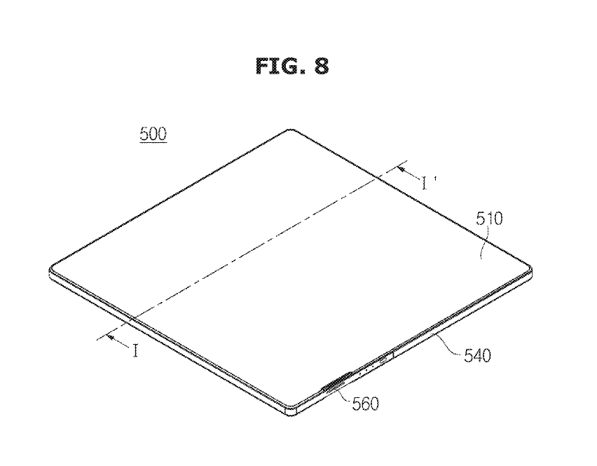

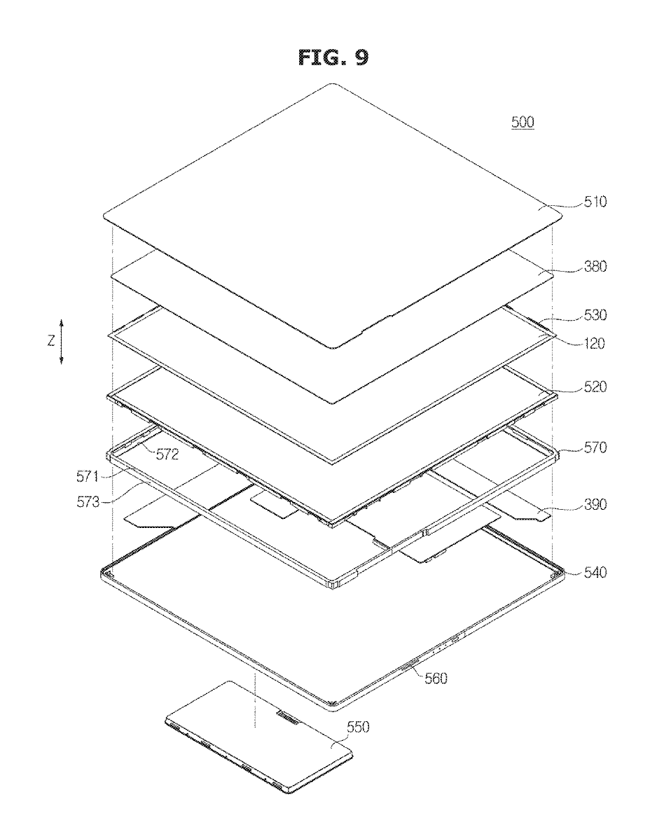

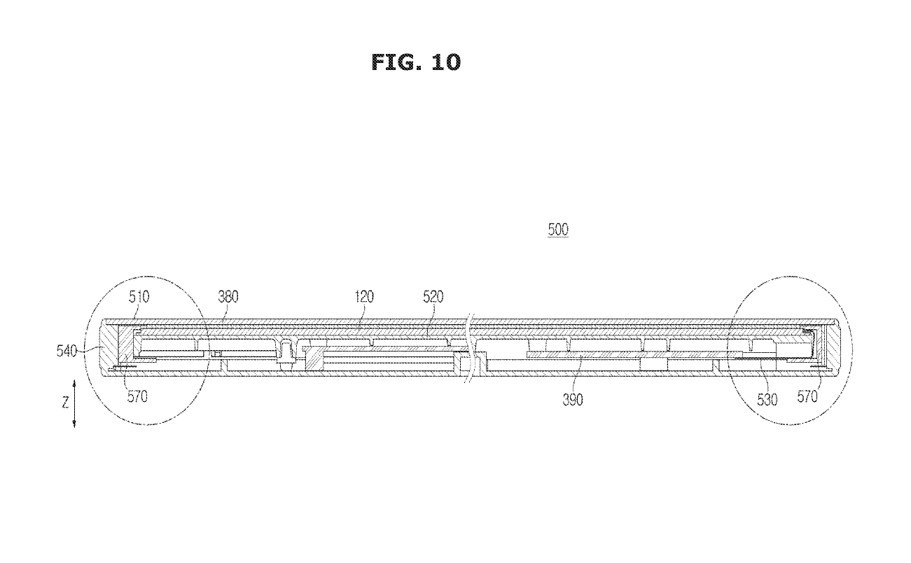

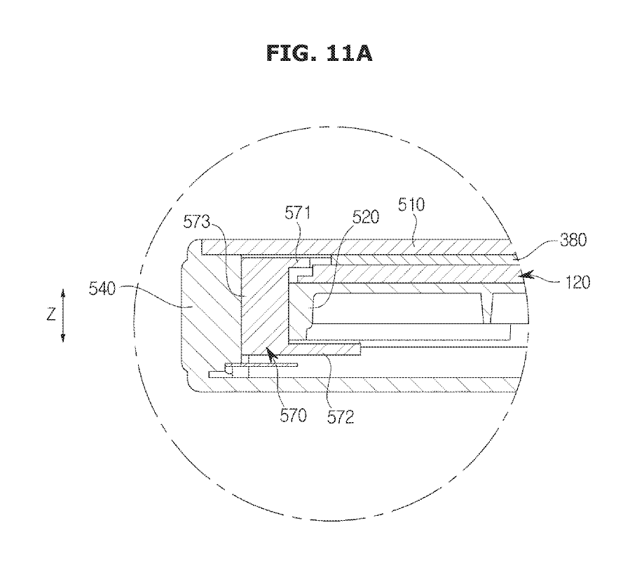

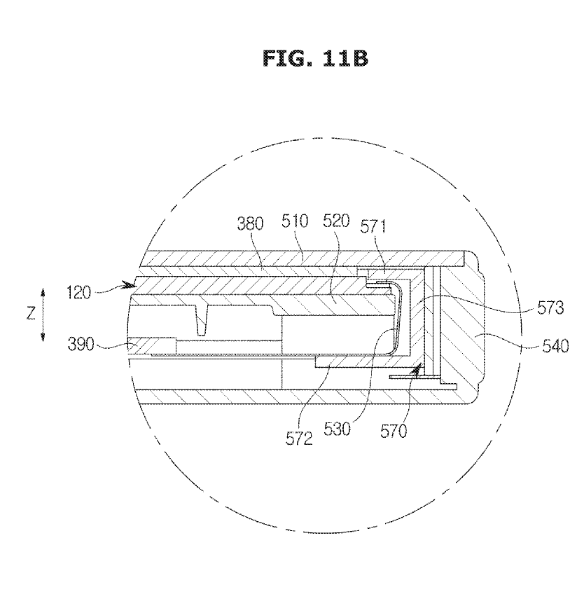

FIG. 8 illustrates a perspective view of an X-ray detector according to a third embodiment of the present disclosure, FIG. 9 illustrates an exploded perspective view of the X-ray detector according to the third embodiment of the present disclosure, FIG. 10 illustrates a cross-sectional view of the X-ray detector of FIG. 8, taken along line I-I′, and FIG. 11A illustrates an enlarged cross-sectional view of a portion surrounded by a right circle in the X-ray detector of FIG. 10. FIG. 11B illustrates an enlarged cross-sectional view of a portion surrounded by a left circle in the X-ray detector of FIG. 10. Hereinafter, the same description as that described above with reference to FIGS. 3 to 6 will be omitted. Also, the same components as those described above with reference to FIGS. 3 to 6 will be assigned the same reference numerals. FIG. 9 schematically shows a structure of a bottom frame 540.

As shown in FIGS. 8 to 11B, an X-ray detector 500 may include the scintillator 380. The scintillator 380 may include a phosphor. The scintillator 380 may convert incident X-rays into visible light. On one surface of the scintillator 380, a cover (not shown) may be disposed to protect the scintillator 380. The scintillator 380 may be formed of a metal material such as aluminum.

The X-ray detector 500 may further include a top fame 510. The top frame 510 may be disposed on the scintillator 380. The top frame 510 may be a carbon plate. On one surface of the top frame 510, a deco sheet (not shown) may be further provided.

The X-ray detector 500 may further include the sensing panel 120. The sensing panel 120 may include a plurality of pixels (not shown), and each pixel may include a thin-film transistor and a photoelectric conversion device such as a photodiode. The sensing panel 120 may read out the intensity of light passed through the scintillator 380 (see FIG. 2) for each pixel. The sensing panel 120 may include an electrical circuit to transfer outputs from the photoelectric conversion device to the outside.

The X-ray detector 500 may further include a middle block 520. The sensing panel 120 may be rested on the middle block 520. In other words, the sensing panel 120 may be disposed on the middle block 520. For example, the sensing panel 120 may be bonded on the middle block 520 to face the top plate 510.

The X-ray detector 500 may further include the circuit board 390. The circuit board 390 may be disposed on the inner surface of the bottom frame 540 to face the middle block 520. However, the circuit board 390 may be disposed at any other position. The circuit board 390 may perform operation for acquiring an image of an object using data acquired based on signals read out by the sensing panel 120. The circuit board 390 may be accommodated in the inside of the X-ray detector 500 in order to control driving of the X-ray detector 500. The circuit board 390 may include memory and an operator. The memory may store shadow information of an object according to an incidence angle of X-rays, and the operator may calculate an incidence angle of X-rays based on a shadow shape of an object formed on the sensing panel 120 and shadow information stored in the memory. The memory and the operator may be disposed outside the X-ray detector 500.

The X-ray detector 500 may further include a flexible circuit board 530. The sensing panel 120 may be electrically connected to the circuit board 390. More specifically, the sensing panel 120 may be connected to the circuit board 390 by the flexible circuit board 530. On the flexible circuit board 530, a read-out terminal may be disposed to read out information of the sensing panel 120.

The X-ray detector 500 may further include the top frame 510 and the bottom frame 540 that are coupled with each other to form the outer appearance of the X-ray detector 500. The top frame 510 may form the upper outer appearance of the X-ray detector 500. The bottom frame 540 may form the side and bottom outer appearances of the X-ray detector 500.

Accommodating space may be formed in the inside of the X-ray detector 500. The middle block 520, the sensing panel 120, the scintillator 380, the circuit board 390, and a cushion 570 may be accommodated in the accommodating space. In other words, the top frame 510 may be coupled with the bottom frame 540 to form accommodating space for accommodating the middle block 520, the sensing panel 120, the scintillator 380, the circuit board 390, and the cushion 570 therein. The bottom frame 540 and the top frame 510 may be formed of the same material.

The X-ray detector 500 may further include a battery 550. The battery 550 may be detachably coupled with the bottom frame 540.

The X-ray detector 500 may further include a terminal 560 with which a coupling module is coupled. The terminal 560 may be disposed in the X-ray detector 500 so that the coupling module can be coupled with the terminal 560. In other words, the terminal 560 may be disposed in the X-ray detector 500 so that the coupling module electrically connected to the circuit board 390 can be coupled with the terminal 560. That is, the coupling module may be coupled with the terminal 560 to be electrically connected to the circuit board 390. More specifically, the terminal 560 may be formed at one side of the bottom frame 540.

The X-ray detector 500 may further include the cushion 570. The cushion 570 may absorb an external impact applied to the X-ray detector 500 to prevent the external impact from being transferred to the sensing panel 120. The cushion 570 may be in the shape of a picture frame. In other words, the cushion 570 may have a cross section in the shape of a rectangle whose one side opens. However, the cushion 570 may be formed in another shape.

The cushion 570 may be formed of an elastic material. The elastic material may be rubber, silicon, or the like, although not limited to these.

The cushion 570 may be disposed between the middle block 520 and the bottom frame 540 to prevent the middle block 520 from directly contacting the bottom frame 540. The cushion 570 may be in close contact with at least one of the middle block 520 and the bottom frame 540. The cushion 570 may be disposed between the middle block 520 and the bottom frame 540 along the edges of the middle block 520. The cushion 570 may be disposed between the middle block 520 and the bottom frame 540 discontinuously along the edges of the middle block 520 in order not to interfere with an electrical connection between the coupling module and the circuit board 390.

The cushion 570 may include a first wall 571 extending toward one direction of the thickness directions Z of the X-ray detector 500. More specifically, the first wall 571 may extend toward the top frame 510.

The cushion 570 may include a second wall 572 extending toward the other direction of the thickness directions Z of the X-ray detector 500. More specifically, the second wall 572 may extend toward the bottom surface of the bottom frame 540.

The cushion 570 may further include a third wall 573 connecting the first wall 571 to the second wall 572 and facing the lateral surface of the bottom frame 540.

In other words, the cushion 570 may include the third wall 573 facing the lateral surface of the bottom frame 540. Also, the cushion 570 may further include the first wall 571 protruding in the inside direction of the X-ray detector 500 from the third wall 573 to be adjacent to the top frame 510. Also, the cushion 570 may further include the second wall 572 protruding in the inside direction of the X-ray detector 500 from the third wall 573 to be adjacent to the bottom surface of the bottom frame 540. The second wall 572 may further protrude than the first wall 571.

One wall of the cushion 570 may be in close contact with one surface of the bottom frame 540. More specifically, the third wall 573 of the cushion 570 may be in close contact with the lateral surface of the bottom frame 540.

The middle block 520 may be disposed in the inside of the cushion 570. At least one portion of the middle block 520 may be in close contact with the cushion 570.

The cushion 570 may be positioned in the inside of the bottom frame 540 to surround the middle block 520 and the sensing panel 120 disposed on the middle block 520.

As such, the cushion 570 may prevent the middle block 520 and the sensing panel from directly contacting the bottom frame 540 and the top frame 510, thereby preventing an external impact from being transferred to the middle block 520 and the sensing panel 120.

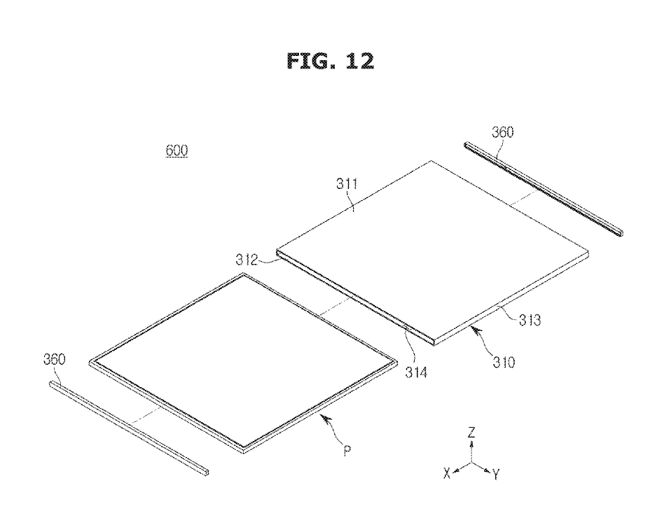

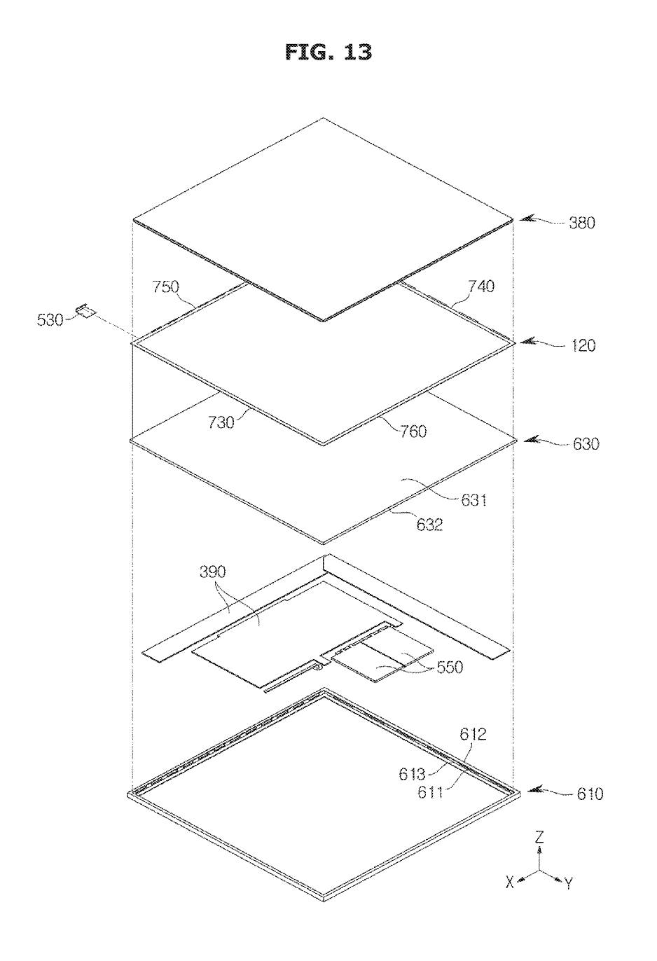

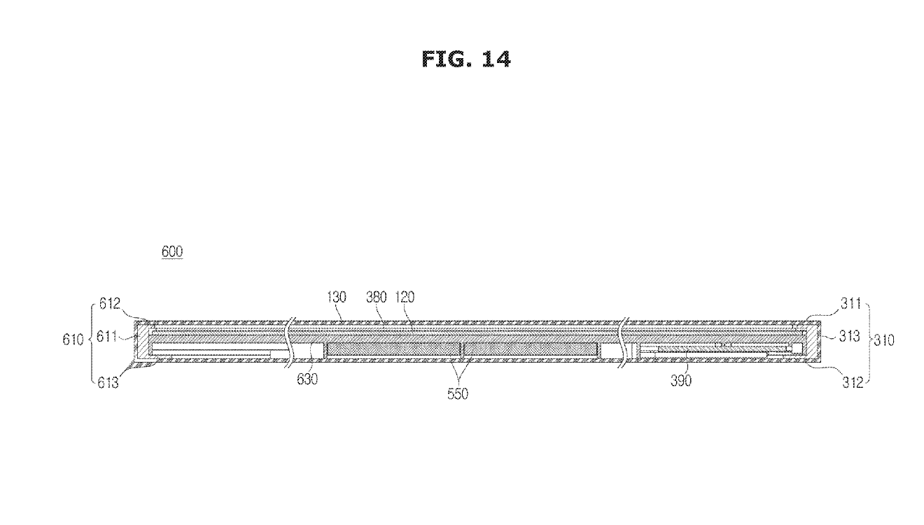

FIG. 12 illustrates an exploded perspective view showing some components of an X-ray detector according to a fourth embodiment of the present disclosure, and FIG. 13 illustrates an exploded perspective view of a portion P of FIG. 12. FIG. 14 illustrates a cross-sectional view of the X-ray detector of FIG. 12. An X-ray detector 600 according to the fourth embodiment of the present disclosure may have the same outer appearance as that of the X-ray detector 300 according to the first embodiment of the present disclosure as shown in FIG. 3. In FIG. 13, reference numerals 730, 740, and 750 and 760 indicate the front surface, the rear surface, and the plurality of lateral surfaces of the sensing panel 120, respectively.

As shown in FIGS. 12, 13, and 14, the X-ray detector 600 may detect X-rays irradiated from the X-ray source 70.

The X-ray detector 600 may include the case 310. The case 310 may form the outer appearance of the X-ray detector 600.

The case 310 may include the first frame 311. On the first frame 311, an incident surface 130 to which X-rays are incident may be formed.

The case 310 may further include the second frame 312. The second frame 312 may be opposite to the first frame 311.

The case 310 may further include the plurality of third frames 313. The third frames 313 may form the outer appearance of the case 310, together with the first frame 311 and the second frame 312. The plurality of third frames 313 may connect the first frame 311 to the second frame 312. More specifically, the plurality of third frames 313 may connect the first frame 311 to the second frame 312 in the thickness directions Z of the X-ray detector 300. In other words, the plurality of third frames 313 may form a side external appearance of the X-ray detector 600.

The case 310 may have the at least one opening 314. For example, the case 310 of the X-ray detector 600 according to the fourth embodiment of the present disclosure may have the plurality of openings 314 that are opposite to each other in the direction X in which a frame 630 is inserted. The case 310 may be in the shape of a box, wherein a plurality of sides facing each other open.

The case 310 may be formed of CFRP, although not limited to this.

The X-ray detector 600 may further include the frame 630. The frame 630 may be detachably inserted into the inside of the case 310 through the at least one opening 314.

The frame 630 may include a first surface 631 corresponding to the first frame 311. The sensing panel 120 and the scintillator 380 may be disposed on the first surface 631 of the frame 630.

The frame 630 may include a second surface 632 corresponding to the second frame 312. The circuit board 390 may be disposed on the second surface 632 of the frame 630. Also, the battery 550 may be disposed on the second surface 632 of the frame 630. In other words, the circuit board 390 and the battery 550 may be mounted on the second surface 632 of the frame 630. However, the circuit board 390 and the battery 550 may be mounted at any other location, instead of the second surface 632 of the frame 630.

The X-ray detector 600 may further include the scintillator 380. The scintillator 380 may include a phosphor. The scintillator 380 may convert incident X-rays into visible light. On one surface of the scintillator 380, a cover (not shown) may be disposed to protect the scintillator 380. The scintillator 380 may be formed of a metal material such as aluminum. The scintillator 380 may be disposed on the sensing panel 120 to face the first frame 311.

The X-ray detector 600 may further include the sensing panel 120. The sensing panel 120 may include a plurality of pixels (not shown), and each pixel may include a thin-film transistor and a photoelectric conversion device such as a photodiode. The sensing panel 120 may read out the intensity of light passed through the scintillator 380 for each pixel. The sensing panel 120 may include an electrical circuit to transfer outputs from the photoelectric conversion device to the outside. The sensing panel 120 may be disposed on the frame 630 to face the first frame 311. More specifically, the sensing panel 120 may be disposed on the first surface 631 of the frame 630 to face the first frame 311. For example, the sensing panel 120 may be bonded on the first surface 631 of the frame 630 to face the first frame 311.

The X-ray detector 600 may further include the circuit board 390. The circuit board 390 may be disposed on the frame 630 to face the second frame 312. More specifically, the circuit board 390 may be disposed on the second surface 632 of the frame 630 to face the second frame 312. The circuit board 390 may be mounted on the second surface 632 of the frame 630. However, the circuit board 390 may be positioned at any other location, instead of the second surface 632 of the frame 630. The circuit board 390 may perform operation for acquiring an image of an object using data acquired based on signals read out by the sensing panel 120. The circuit board 390 may be accommodated in the inside of the case 310 in order to control driving of the X-ray detector 600. The circuit board 390 may include memory and an operator. The memory may store shadow information of an object according to an incidence angle of X-rays, and the operator may calculate an incidence angle of X-rays based on a shadow shape of an object formed on the sensing panel 120 and shadow information stored in the memory.

The X-ray detector 600 may further include the flexible circuit board 530. The sensing panel 120 may be electrically connected to the circuit board 390. More specifically, the sensing panel 120 may be connected to the circuit board 390 by the flexible circuit board 530. On the flexible circuit board 530, a read-out terminal (not shown) may be disposed to read out information of the sensing panel 120.

The X-ray detector 600 may further include the battery 550. The battery 550 may be disposed in the inside of the case 310. More specifically, the battery 550 may be mounted on the second surface 632 of the frame 630 to face the second frame 312. However, the battery 550 may be positioned at any other location, instead of the second surface 632 of the frame 630.

The X-ray detector 600 may further include a buffer member 610. The buffer member 610 may absorb an external impact applied to the X-ray detector 600 to prevent the external impact from being transferred to the sensing panel 120. The buffer member 610 may surround at least one portion of the edges of the sensing panel 120. Preferably, the buffer member 610 may surround all the edges of the sensing panel 120. The edges of the sensing panel 120 may be vulnerable to impacts. The buffer member 610 may be disposed outside the sensing panel 120 in such a way to surround the edges of the sensing panel 120, thereby preventing an external impact from being directly transferred to the sensing panel 120. The buffer member 610 may be formed of an elastic material. For example, the buffer member 610 may be formed of silicon, rubber, or the like.

The buffer member 610 may have a shape that is similar to a picture frame.

The buffer member 610 may include a body 611, a first support portion 613 bent from the body 611 toward the inside direction of the X-ray detector 600 to be positioned on the sensing panel 120, and a second support portion 613 bent from the body 611 toward the inside direction of the X-ray detector 600 to be positioned beneath the sensing panel 120. The body 611, the first support portion 612, and the second support portion 613 may be integrated into one body. Also, the first support portion 612 and the second support portion 613 may be aligned in parallel, although not limited to this.

The frame 630, the sensing panel 120, and the scintillator 380 may be disposed in the inside of the buffer member 610. That is, the buffer member 610 may be disposed outside the frame 630, the sensing panel 120, and the scintillator 380 in such a way to be in close contact with the edges of the frame 630, the sensing panel 120, and the scintillator 380. More specifically, the frame 630 may be rested on the second support portion 613 of the buffer member 610. The scintillator 380 may be disposed beneath the first support portion 612 of the buffer member 610, and supported by the first support portion 612. As such, since the buffer member 610 is disposed to surround all the edges of the frame 630, the sensing panel 120, and the scintillator 380, it is possible to more effectively prevent an external impact from being directly transferred to the frame 630 and the scintillator 380, as well as the sensing panel 120.

The X-ray detector 600 may further include the at least one cap 360.

The at least one cap 360 may open or close the at least one opening 314 of the case 310.

Also, the at least one cap 360 may be detachably coupled with the at least one opening 314.

The X-ray detector 600 according to the fourth embodiment of the present disclosure may include the plurality of caps 360 configured to open or close the plurality of openings 314 of the case 310. The plurality of openings 314 may be formed to be opposite to each other in the direction X in which the frame 320 is inserted.

The X-ray detector 600 may further include the terminal (not shown) with which the coupling module (not shown) is coupled. The terminal may be disposed in the X-ray detector 600 such that the coupling module can be coupled with the terminal. In other words, the terminal may be disposed in the X-ray detector 600 such that the coupling module electrically connected to the circuit board 390 can be coupled with the terminal. That is, the coupling module may be coupled with the terminal to be electrically connected to the circuit board 390. The terminal may be disposed at one side of the case 310, although not limited to this. That is, the terminal may be disposed at any other location.

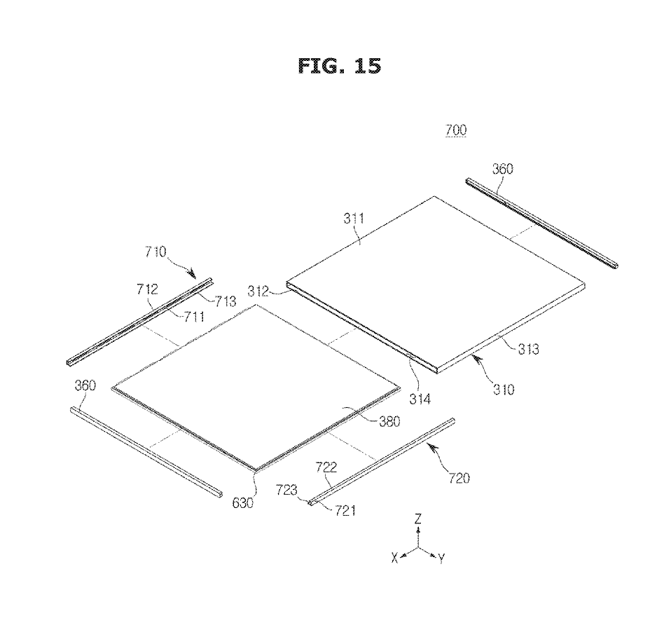

FIG. 15 illustrates an exploded perspective view showing some components of an X-ray detector according to a fifth embodiment of the present disclosure. An X-ray detector 700 according to the fifth embodiment of the present disclosure may have the same outer appearance as that of the X-ray detector 300 according to the first embodiment of the present disclosure as shown in FIG. 3. Hereinafter, the same description as that described above in regard of the X-ray detector 600 according to the fourth embodiment of the present disclosure will be omitted. Also, reference numerals although not defined below will be understood from the above descriptions about the X-ray detector 600 according to the fourth embodiment of the present disclosure.

As shown in FIG. 15, the X-ray detector 700 may include buffer members 710 and 720. The buffer members 710 and 720 may absorb an external impact applied to the X-ray detector 700 to prevent the external impact from being transferred to the sensing panel 120. The buffer members 710 and 720 may surround at least one part of the edges of the sensing panel 120. Preferably, the buffer members 710 and 720 may surround a part of the edges of the sensing panel 120. The edges of the sensing panel 120 may be vulnerable to impacts. The buffer members 710 and 720 may be disposed outside the sensing panel 120 in such a way to surround the edges of the sensing panel 120, thereby preventing an external impact from being directly transferred to the sensing panel 120. The buffer members 710 and 720 may be formed of an elastic material. For example, the buffer members 710 and 720 may be formed of silicon, rubber, or the like.

The buffer members 710 and 720 may include a first buffer member 710 surrounding one edge of the sensing panel 120, and a second buffer member 720 surrounding the other edge of the sensing panel 120. The first buffer member 710 may be opposite to the second buffer member 720.

The sensing panel 120 may include a front surface 730 located at the front in the direction X in which the frame 630 is inserted, a rear surface 740 located at the rear in the direction X in which the frame 630 is inserted, and a plurality of side surfaces 750 and 760 connecting the front surface 73 to the rear surface 740. In other words, the sensing panel 120 may include the front surface 730 and the rear surface 740 facing the plurality of caps 360, respectively, and the plurality of side surfaces 750 and 760 facing the plurality of third frames 313 of the case 310, respectively.

The first buffer member 710 may surround one of the plurality of side surfaces 750 and 760 of the sensing panel 120. The second buffer member 720 may surround the other one of the plurality of side surfaces 750 and 760 of the sensing panel 120. The first buffer member 710 may be coupled with the outer portion of one of the plurality of side surfaces 750 and 760 of the sensing panel 120. The second buffer member 720 may be coupled with the outer portion of the other one of the plurality of side surfaces 750 and 760 of the sensing panel 120.

The first buffer member 710 and the second buffer member 720 may include bodies 711 and 721, first support portions 712 and 722 bent from the bodies 711 and 721 in the inside direction of the X-ray detector 700 to be positioned on the sensing panel 120, and second support portions 713 and 723 bent from the bodies 711 and 721 in the inside direction of the X-ray detector 700 to be positioned beneath the sensing panel 120, respectively. The bodies 711 and 721, the first support portions 712 and 722, and the third support portions 713 and 723 may be integrated into one body. Also, the first support portions 712 and 722 and the second support portions 713 and 723 may be aligned in parallel, although not limited to this.

The first buffer member 710 and the second buffer member 720 may be disposed outside the frame 630, the sensing panel 120, and the scintillator 380 to be in close contact with the edges of the frame 630, the sensing panel 120, and the scintillator 380. More specifically, the frame 630 may be rested on the second support portions 713 and 723 of the first buffer member 710 and the second buffer member 720. The scintillator 380 may be disposed beneath the first support portions 712 and 722 of the first buffer member 710 and the second buffer member 720, and supported by the first support portions 712 and 722. Both sides of the case 310 in which the plurality of openings 314 are formed may be ensured or maintained with a predetermined level of rigidity by the plurality of caps 360 coupled with the plurality of openings 314. However, the plurality of third frames 313 of the case 310 may have relatively weaker rigidity than both sides of the case 310 in which the plurality of openings 314 are formed. Accordingly, the plurality of third frames 313 of the case 310 may be relatively easily deformed by an external impact than the other portion of the case 310. Such deformation of the plurality of third frames 313 of the case 310 may influence the sensing panel 120, and accordingly, the first buffer member 710 and the second buffer member 720 may be disposed in order to minimize such influence.

Therefore, by disposing the impact absorbing member in the inside of the case, it is possible to prevent a vulnerable portion of the X-ray detector from being broken or damaged by an external impact.

By disposing the sensing panel in the inside of the case in such a way to be spaced apart from the case and the frame, it is possible to prevent an external impact from being directly transferred to the sensing panel.

Since the buffer member is in close contact with the case and the frame in such a way to surround the plurality of surfaces of the frame, the buffer member can more effectively absorb an external impact.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. An X-ray detector for detecting X-rays irradiated from an X-ray source, the X-ray detector comprising:
a case having at least one opening;
a sensing panel configured to convert the X-rays irradiated from the X-ray source into an electrical signal;
a frame detachably inserted into an inside of the case through the at least one opening, the frame including a body on which the sensing panel is disposed and a plurality of legs extending from the body in a first direction of the X-ray detector; and
a plurality of buffer members disposed between the plurality of legs and the case in close contact with the plurality of legs and the case while surrounding a plurality of surfaces of each of the plurality of legs.

2. The X-ray detector according to claim 1, wherein the plurality of buffer members surround a plurality of surfaces of the plurality of legs where the plurality of surfaces face the case.

3. The X-ray detector according to claim 2, wherein each of the plurality of legs includes:
a first surface facing one direction of the thickness directions of the X-ray detector;
a second surface facing the other direction of the thickness directions of the X-ray detector; and
a third surface connecting the first surface to the second surface, the third surface facing the case,
wherein the plurality of buffer members are configured to surround the first surface, the second surface, and the third surface.

4. The X-ray detector according to claim 1, wherein the sensing panel is disposed on the body so that the sensing panel is spaced apart from the frame.

5. The X-ray detector according to claim 4, wherein the body includes a sensing panel corresponding surface facing the sensing panel,
the X-ray detector further comprising at least one support member disposed between the sensing panel and the sensing panel corresponding surface, the at least one support member having an elastic material.

6. The X-ray detector according to claim 4, further comprising a plurality of cushion members disposed between the plurality of legs and the sensing panel, the plurality of cushion members having an elastic material.

7. The X-ray detector according to claim 1, further comprising at least one cap detachably coupled with the at least one opening to open or close the at least one opening of the case.

8. The X-ray detector according to claim 1, wherein the case has a plurality of openings that are opposite to each other in a second direction in which the frame is inserted,
the X-ray detector further comprises a plurality of caps configured to open or close the plurality of openings of the case, and
one cap of the plurality of caps is integrated into the frame.

9. An X-ray detector of detecting X-rays, comprising:
a case having at least one opening;
a frame detachably inserted into the inside of the case through the at least one opening;
a sensing panel configured to convert the X-rays into an electrical signal and disposed on the frame so that the sensing panel is spaced apart from the case and the frame; and
a buffer member configured to prevent the frame from directly contacting the case, the buffer member is disposed between the case and the frame in close contact with the case and the frame while surrounding a plurality of surfaces of the frame.

10. The X-ray detector according to claim 9, wherein the frame comprises:
a body on which the sensing panel is disposed; and
a plurality of legs extending from edges of the body in a first direction of the X-ray detector,
wherein the buffer member is disposed between the case and the plurality of legs in close contact with the case and the plurality of legs while surrounding a plurality of surfaces of each of the plurality of legs.

11. The X-ray detector according to claim 9, wherein the case comprises:
a first frame forming an incident surface onto which the X-rays are incident;
a second frame facing the first frame; and
a plurality of third frames forming an outer appearance of the case, together with the first frame and the second frame,
wherein the buffer member surrounds a plurality of surfaces of the frame in close contact with the first frame, the second frame, and the plurality of third frames.

12. The X-ray detector according to claim 10, further comprising a support member having at least one part disposed between the sensing panel and the body, the support member having an elastic material.

13. The X-ray detector according to claim 12, wherein an incision is formed in the body, and a part of the support member is inserted into the incision.

14. The X-ray detector according to claim 10, further comprising a cushion member disposed between the plurality of legs and the sensing panel along at least one edge of the sensing panel, the cushion member having an elastic material.

15. The X-ray detector according to claim 9, further comprising at least one cap configured to open or close the at least one opening of the case.

16. An X-ray detector of detecting X-rays, comprising:
a case;
a frame disposed in the inside of the case and spaced apart from the case;
a sensing panel configured to convert the X-rays into an electrical signal, the sensing panel is disposed on the frame so that the sensing panel is spaced apart from the case and the frame; and
an impact absorbing member disposed in the inside of the case, the impact absorbing member is configured to absorb an external impact,
wherein the impact absorbing member comprises:
a first impact absorbing member disposed between the frame and the case; and
a second impact absorbing member disposed between the frame and the sensing panel to be positioned in the inside of the first impact absorbing member in a first direction of the X-ray detector.

17. The X-ray detector according to claim 16, wherein the first impact absorbing member is disposed between the case and the frame in close contact with the case and the frame while surrounding a plurality of surfaces of the frame.

18. The X-ray detector according to claim 17, wherein the case comprises:
a first frame forming an incident surface onto which the X-rays are incident;
a second frame facing the first frame; and
a plurality of third frames forming an outer appearance of the case, together with the first frame and the second frame,
wherein the first impact absorbing member surrounds the plurality of surfaces of the frame in close contact with the first frame, the second frame, and the plurality of third frames.

19. The X-ray detector according to claim 16, wherein the impact absorbing member further comprises a third impact absorbing member having at least one part disposed between the frame and the sensing panel in a second direction of the X-ray detector.

20. The X-ray detector according to claim 16, further comprising at least one cap configured to open or close at least one opening of the case.
